(19) United States
(12) Patent Application Publication
(10) Pub. No.: US 2016/0243064 A1
(43) Pub. Date: Aug. 25, 2016

(54) NOVEL ORAL PHARMACEUTICAL COMPOSITIONS OF TREPROSTINIL

(71) Applicant: Gavis Pharmaceuticals, Somerset, NJ (US)

(72) Inventors: Aman Trehan, Somerset, NJ (US); James Garegnani, Somerset, NJ (US)

(73) Assignee: Gavis Pharmaceuticals, Somerset, NJ (US)

(21) Appl. No.: 15/049,335

(22) Filed: Feb. 22, 2016

Related U.S. Application Data
(60) Provisional application No. 62/119,159, filed on Feb. 21, 2015.

(51) Int. Cl. A61K 31/192; A61K 9/00

(52) U.S. Cl. CPC A61K 31/192; A61K 9/0053; A61K 9/0019

(57) ABSTRACT

Oral pharmaceutical compositions of treprostinil, a prodrug, or a pharmaceutically acceptable salt thereof are provided. The compositions possess absolute oral bioavailability of greater than 10% and achieve circulating concentrations when administered orally. Methods of treating prostacyclin responsive conditions including pulmonary arterial hypertension (PAH) by administering such compositions also are provided.

### CROSS-REFERENCE TO RELATED APPLICATIONS

This application is a nonprovisional application claiming the benefit priority of U.S. Provisional Application Ser. No. 62/119,159, filed Feb. 21, 2015, the contents of which is incorporated herein in its entirety by reference.

### TECHNICAL FIELD OF THE INVENTION

The present invention is directed to novel oral pharmaceutical compositions of treprostinil, a prodrug, or a pharmaceutically acceptable salt thereof. The compositions of the present invention possess oral bioavailability and achieve circulating concentrations when administered orally. The compositions comprise treprostinil free acid, a prodrug, or a pharmaceutically acceptable salt thereof excluding diethanolamine salt. The invention also relates to methods of treating prostacyclin responsive conditions including pulmonary arterial hypertension (PAH) by administering such compositions.

### DESCRIPTION OF THE RELATED ART

Pulmonary arterial hypertension (PAH) is an increase in blood pressure in the pulmonary artery, pulmonary vein, or pulmonary capillaries, leading to shortness of breath, dizziness, fainting, and other symptoms, all of which are exacerbated by exertion. PAH can be a severe disease with a markedly decreased exercise tolerance and heart failure. It is an orphan disease with an incidence of about 2-3 per million per year and a prevalence of about 15 per million. Median survival of patients with untreated PAH is in the range of 2-3 years from time of diagnosis, with the cause of death usually being right ventricular failure.

Pulmonary arterial hypertension involves the vasoconstriction or tightening of blood vessels connected to and within the lungs. Over time, fibrosis causes the affected blood vessels to become both stiffer and thicker which further increases the blood pressure within the lungs and impairs their blood flow. In addition, the increased workload of the heart causes hypertrophy of the right ventricle which ultimately causes right heart failure. As the blood flowing through the lungs decreases, the left side of the heart receives less blood and thus oxygen supply is below the required level, especially during physical activity.

A number of agents have been introduced for the treatment of PAH of which prostacyclins are commonly considered to be the most effective. Prostacyclin derivatives are useful pharmaceutical compounds possessing activities such as platelet aggregation inhibition, gastric secretion reduction, lesion inhibition, and bronchodilation.

One prostacyclin is Epoprostenol which is a synthetic prostacyclin and marketed as Flolan® (GlaxoSmithKline). It is given to patients via continuous infusion and requires a semi-permanent central venous catheter which can cause sepsis and thrombosis. Flolan® is unstable, and therefore has to be kept on ice during administration. Since it has a half-life of only 3 to 5 minutes, the infusion has to be continuous night and day and any interruption can be fatal. Thus, treatment of PAH with Flolan® is a huge burden for the patient.

Another prostacyclin, Iloprost (Ilomedin) which is marketed as Ventavis® (Bayer), was the only inhaled form of prostacyclin approved for use in the US and Europe until the inhaled form of treprostinil was approved by the FDA in July 2009 which is marketed under the trade name TYVASO® (United Therapeutics).

Inhaled prostacyclin suffer from the drawback of not providing fully efficacious plasma levels of drug throughout the dosing period, making inhaled therapy less desired in severe patients.

Therefore, there was a need to develop other prostanoids, as has been described for example in U.S. Pat. No. 4,306,075A and EP 159784 B1. One such prostaglandin is treprostinil. Although the half-life of treprostinil is 4 hours, treprostinil is still required to be administered as a continuous subcutaneous infusion or continuous intravenous infusion via an infusion pump that the patient must wear at all times.

Treprostinil is a synthetic analogue of prostacyclin and the active ingredient in Remodulin® and Tyvaso®. Treprostinil, and other prostacyclin derivatives may be prepared as described in Moriarty, et al in J. Org. Chem. 2004, 69, 1890-1902, Drug of the Future, 2001, 26(4), 364-374, U.S. Pat. Nos. 6,441,245; 6,528,688; 6,700,025; 6,809,223; 6,756,117; 8,461,393; 8,481,782; 8,242,305; and 8,497,393; U.S. Patent Publication Nos. 2012-0190888 and 2012-0197041; and PCT Publication No. WO2012/009816.

Treprostinil has the following chemical structure:

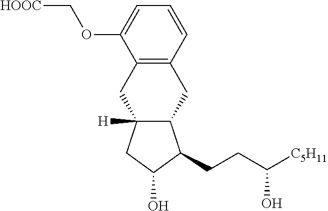

Various uses and/or various forms of treprostinil are disclosed, for example, in U.S. Pat. Nos. 5,153,222; 5,234,953; 6,521,212; 6,756,033; 6,803,386; 7,199,157; 6,054,486; 7,384,978; 7,879,909; 8,563,614; 8,232,316; 8,609,728; 8,350,079; 8,349,892; 7,999,007; 8,658,694; and 8,653,137; U.S. Patent Publication Nos. 2005/0165111, 2009/0036465, 2008/0200449, 2010/0076083, 2012?0216801, 2008/0280986, 2009-0124697, and 2013/0261187; PCT publication no. WO00/57701; U.S. Provisional Applications Nos. 61/781,303 filed Mar. 14, 2013 and 61/805,048 filed Mar. 25, 2013. The teachings of the aforementioned references are incorporated by reference to show how to practice the embodiments of the present invention.

Parenteral therapy with treprostinil suffers from a complex side effect profile, astronomical cost of treatment, and compliance issues especially in geriatric and paediatric patients.

Recently, an extended-release oral tablet formulation of treprostinil has been approved in the United States and sold under the brand name Orenitram™. The product contains the diethanolamine salt of treprostinil. According to the product label, the absolute oral bioavailability of Orinetram™ tablet is approximately 17%.

U.S. Pat. Nos. 7,544,713; 7,417,070; 8,536,363; 8,410,169 and 8,252,839 disclose the diethanolamine salt of treprostinil. The patents teach that treprostinil as a free acid has an oral absolute bioavailability of less than 10%. The patents further teach that the diethanolamine salt of treprostinil possesses three properties which are not available with treprostinil as free acid and other known salt choices: a relatively high melting temperature, a relatively high aqueous solubility, and a relatively low hygroscopicity. These three properties are essential from an oral formulation perspective.

Though oral preparations of treprostinil and its sodium salt have been disclosed (e.g., U.S. Pat. Publication Nos. 20050165111 and 20050282903, and U.S. Pat. Nos. 5,153,222, 5,028,628, and 6,054,486), none of these publications addresses an issue of oral bioavailability of treprostinil free acid or its salts including the sodium salt nor does any reference teach formulations of treprostinil free acid or its salts that exhibits oral bioavailability.

In sum, treprostinil is of great importance from a medicinal point of view. Orinetram™ tablet is the only product of treprostinil currently available in the market which is suitable for oral administration. Further, according to the prior art, diethanolamine is the only salt suitable to use in a formulation meant for oral administration due to its desired characteristics. Thus there still exists a need for new compositions of treprostinil which are suitable for oral administration and that contain stable forms of treprostinil including the various known forms which impart advantages, for example, in manufacturing, storage, and/or in formulation. Such compositions also should provide a viable and economical treatment option for management of prostacyclin responsive conditions, and preferably for PAH.

### SUMMARY OF THE INVENTION

The present invention provides oral pharmaceutical compositions of treprostinil, a prodrug, or a pharmaceutically acceptable salt thereof.

In one aspect, the invention provides an oral pharmaceutical composition comprising treprostinil free acid.

In another aspect, the invention provides an oral pharmaceutical composition comprising a pharmaceutically acceptable salt of treprostinil, excluding the diethanolamine salt.

In another aspect, the invention provides an oral pharmaceutical composition comprising a pharmaceutically acceptable salt of treprostinil selected from a group consisting of ammonium salts (such as quaternary ammonium salts), alkali metal salts such as those of sodium and potassium, alkaline earth metal salts such as those of calcium and magnesium, salts with organic bases such as dicyclohexylamine and N-methyl-D-glucamine, and salts with amino acids such as arginine and lysine.

In another aspect, the invention provides an oral pharmaceutical composition comprising a pharmaceutically acceptable salt of treprostinil selected from a group consisting of calcium, ethylenediamine, choline, hydroxymethyl aminomethane (TRIS), sodium, potassium, glucamine, benzathine, and procaine.

In another aspect, the invention provides an oral pharmaceutical composition comprising treprostinil sodium.

The present invention provides an oral pharmaceutical composition comprising a prodrug of treprostinil.

In another aspect, the invention provides an oral pharmaceutical composition of treprostinil, a prodrug, or a pharmaceutically acceptable salt thereof, excluding the diethanolamine salt; wherein oral bioavailability of the composition is equal to or greater than the oral bioavailability of the treprostinil diethanolamine composition.

In another aspect, the invention provides an oral pharmaceutical composition of treprostinil, a prodrug, or a pharmaceutically acceptable salt thereof, excluding the diethanolamine salt; wherein oral bioavailability of the composition is at least 10%.

In another aspect, the invention provides an oral pharmaceutical composition of treprostinil, a prodrug, or a pharmaceutically acceptable salt thereof, excluding the diethanolamine salt; wherein oral bioavailability of the composition is at least 17%.

In another aspect, the invention provides an oral extended release tablet comprising treprostinil, a prodrug, or a pharmaceutically acceptable salt thereof, excluding the diethanolamine salt.

In another aspect, the invention provides an oral extended release osmotic tablet comprising treprostinil, a prodrug, or a pharmaceutically acceptable salt thereof, excluding the diethanolamine salt, wherein oral bioavailability of the composition is at least 10%.

In another aspect, the invention provides an oral extended release osmotic tablet comprising treprostinil, a prodrug, or a pharmaceutically acceptable salt thereof, excluding the diethanolamine salt, wherein oral bioavailability of the composition is at least 17%.

In another aspect, the invention provides a method of using the oral pharmaceutical composition comprising treprostinil, a prodrug, or a pharmaceutically acceptable salt thereof, excluding diethanolamine; for pulmonary hypertension, ischemic diseases, heart failure, conditions requiring anticoagulation, thrombotic microangiopathy, extracorporeal circulation, central retinal vein occlusion, atherosclerosis, inflammatory diseases, hypertension, reproduction and parturition, cancer or other conditions of unregulated cell growth, cell/tissue preservation and other emerging therapeutic areas where prostacyclin treatment appears to have a beneficial role.

A preferred aspect is a method of treating pulmonary hypertension and/or peripheral vascular disease in a patient comprising orally administering the pharmaceutical composition comprising treprostinil, a prodrug, or a pharmaceutically acceptable salt thereof, excluding the diethanolamine salt to said patient.

In another aspect, the invention provides a method of reducing the side effects or toxicity associated with oral administration of a composition of treprostinil or a salt thereof, the method comprises of orally administering the pharmaceutical composition comprising treprostinil, a prodrug, or a pharmaceutically acceptable salt thereof, excluding the diethanolamine salt.

In another aspect, the invention provides a method of providing a treatment for PAH that is therapeutically comparable to a known method for treating PAH consisting essentially of intravenous or inhalation administration of treprostinil or a pharmaceutically acceptable salt thereof. The comparable method consists essentially of orally administering the pharmaceutical composition comprising treprostinil, a prodrug, or a pharmaceutically acceptable salt thereof, excluding the diethanolamine salt. The comparable method is bioequivalent to the intravenous or inhalation methods.

Still other aspects and advantages of the invention will be apparent from the following detailed description of the invention.

### DETAILED DESCRIPTION OF THE INVENTION

The invention provides for oral pharmaceutical compositions comprising treprostinil, a prodrug, or a pharmaceutically acceptable salt thereof other than the diethanolamine salt. The composition of the invention is characterized by exhibiting oral bioavailability that is at least equivalent to or greater than that exhibited by oral administration of treprostinil diethanolamine composition.

The inventors of the present invention have surprisingly found that it is possible to prepare an alternative pharmaceutical composition of treprostinil which is suitable for oral administration and exhibits bioavailability which is comparable to that exhibited after oral administration of a treprostinil diethanolamine formulation. Particularly, the inventors have found that the composition of the invention comprising treprostinil in the form of free acid, prodrug or a pharmaceutically acceptable salt, such as sodium salt, possess absolute oral bioavailability of greater than 10%, and preferably, at least 17%. It is believed that by judicious selection of pharmaceutical carriers and their use in specific amounts, orally bioavailable compositions of treprostinil free acid, its prodrug or a pharmaceutically acceptable salt can be prepared. Without wishing to be bound by any particular theory, the qualitative and quantitative content of the composition of the invention may be compensating for the melting temperature, aqueous solubility, and/or hygroscopicity of treprostinil free acid, its prodrug, or a pharmaceutically acceptable salt so as to exhibit oral bioavailability of greater than 10%.

For the purpose of present invention, free acid form, prodrug or a pharmaceutically acceptable salt, specifically excluding the diethanolamine salt of treprostinil, may be used.

As used herein, the term “prodrug” means a derivative of treprostinil that can hydrolyze, oxidize, or otherwise react under biological conditions (in vitro or in vivo) to provide an active compound, treprostinil. Examples of prodrugs include, but are not limited to, derivatives of a compound that include biohydrolyzable groups such as biohydrolyzable amides, biohydrolyzable esters, biohydrolyzable carbamates, biohydrolyzable carbonates, biohydrolyzable ureides, and biohydrolyzable phosphate analogues (e.g., monophosphate, diphosphate or triphosphate).

Prodrugs of treprostinil convert to treprostinil after oral administration to a patient. In some embodiments, the prodrugs have little or no activity themselves and only show activity after being converted to treprostinil. In some embodiments, the present compounds were produced by chemically derivatizing treprostinil to make stable esters, and in some instances, the compounds were derivatized from the hydroxyl groups. Prodrugs of treprostinil for use in the present invention can also be provided by modifying the compounds found in U.S. Pat. Nos. 4,306,075 and 5,153,222 in like manner. In an embodiment, the prodrug of treprostinil may be selected from compounds disclosed in U.S. Patent Publication No. 20140296150. In an embodiment, the prodrug of treprostinil is in the form of PEG-linked treprostinil. In an embodiment, the prodrugs of treprostinil are fatty alcohols or lipid nanoparticle linked treprostinil.

The term “treprostinil” as used herein includes both anhydrous and monohydrate form of treprostinil. Various monohydrate forms of treprostinil may be selected from those disclosed in U.S. Pat. No. 8,350,079 and U.S. Application No. 20140275262, the contents of which are incorporated by reference in its entirety for the purposes of the monohydrates forms, their preparation and use.

By “osmotic” delivery system or dosage form herein is meant an elementary osmotic pump (EOP) system. An EOP is very well known to those skilled in the art. This system can be used to test for drugs which are “erratic” or give an incomplete release profile, as defined. See, e.g., Felix Theeuwes, Elementary Osmotic Pump, Journal of Pharmaceutical Sciences, Vol. 64, No. 12, Pp 1987-1991, December 1975.

The terms “bioavailable” or “bioavailability” as used herein refer to the amount of a drug made available to target tissues in a subject through the systemic circulation in the subject's body. In this context, the terms “oral bioavailable” or “oral bioavailability” may specifically refer to the ability of the drug to be absorbed from the gastrointestinal tract into a lymph which will then enter into the bloodstream of an individual such that the drug can be absorbed into organs and tissues in the body.

Bioequivalence is established by comparing pharmacokinetic parameters, for example AUC and Cₘₐₓ, of the present invention with, for example Orenitram® extended release tablets in healthy human subjects. The term “AUC” refers to the area under the time/plasma concentration curve after the administration of the treprostinil dosage form to healthy human subjects. The term “Cₘₐₓ” refers to the maximum concentration of treprostinil in the blood following the administration of the treprostinil dosage form to healthy human subjects.

Bioequivalence is defined to mean the term used by the drug approval agencies, such as the US Food and Drug Administration: “the absence of a significant difference in the rate and extent to which the active ingredient or active moiety in pharmaceutical equivalents or pharmaceutical alternatives becomes available at the site of drug action when administered at the same molar dose under similar conditions in an appropriately designed study.” This is typically understood to mean that the test drug product is within +25% and −20% of the reference drug product for AUC and Cₘₐₓ, for example as explained in the US FDA's various bioequivalence guidance documents for oral tablets and capsules, which are incorporated herein by reference.

The term “pharmaceutically acceptable salt” as used herein means those salts of treprostinil that are safe and effective for pharmaceutical use in mammals and that possess the desired biological activity. Such salts include acid addition salts formed with organic and inorganic acids, such as hydrogen chloride, hydrogen bromide, hydrogen iodide, sulfuric acid, phosphoric acid, acetic acid, glycolic acid, maleic acid, malonic acid, oxalic acid, methane sulfonic acid, trifluoroacetic acid, fumaric acid, succinic acid, tartaric acid, citric acid, benzoic acid, ascorbic acid and the like. Base addition salts may be formed with organic and inorganic bases, such as sodium, ammonia, potassium, calcium, ethanolamine, diethanolamine, N-methylglucamine, choline and the like.

Base addition salts are salts of basic groups present in the specified compounds, and acid addition salts, which are salts of acid groups present in the specific compounds. The acidic or basic groups can be organic or inorganic.

The pharmaceutically acceptable salts of the present invention are preferably base addition salts of treprostinil. Base addition salts include salts formed with inorganic bases and organic bases. The term “inorganic base,” as used herein, has its ordinary meaning as understood by one of ordinary skill in the art and broadly refers to an inorganic compound that can act as a proton acceptor. The term “organic base,” as used herein, also has its ordinary meaning as understood by one of ordinary skill in the art and broadly refers to an organic compound that can act as a proton acceptor.

Examples of inorganic bases that can be used to form base addition salts include, but are not limited to, metal hydroxides, such as lithium hydroxide, sodium hydroxide, and potassium hydroxide; metal amides, such as lithium amide and sodium amide; metal carbonates, such as lithium carbonate, sodium carbonate, and potassium carbonate; and ammonium bases such as ammonium hydroxide and ammonium carbonate.

In a preferred embodiment of the present invention, the inorganic base is selected from metal hydroxides and ammonium hydroxide. Preferred metal hydroxides include sodium hydroxide and potassium hydroxide.

Examples of organic bases that can be used to form base addition salts include, but are not limited to, metal alkoxides, such as lithium, sodium, and potassium alkoxides including lithium methoxide, sodium methoxide, potassium methoxide, lithium ethoxide, sodium ethoxide, potassium ethoxide, and potassium tert-butoxide; quaternary ammonium hydroxides, such as choline hydroxide; and amines including, but not limited to, aliphatic amines (i.e., alkylamines, alkenylamines, alkynylamines, and alicyclic amines), heterocyclic amines, arylamines, heteroarylamines, basic amino acids, amino sugars, and polyamines. Preferred aliphatic amine bases include alkylamines.

In preferred embodiment, pharmaceutically acceptable salts of treprostinil suitable for use in the composition of the present invention include salts derived from bases. Base salts include amine salts, ammonium salts (such as quaternary ammonium salts), alkali metal salts such as those of sodium and potassium, alkaline earth metal salts such as those of calcium and magnesium, salts with organic bases such as dicyclohexylamine and N-methyl-D-glucamine, and salts with amino acids such as arginine and lysine.

Suitable amine salts may be selected from the group consisting of alkylamines, heterocyclic amines, arylamines, heteroarylamines, basic amino acids, amino sugars, polyamines, alkenylamines, alkynylamines, and alicyclic amines.

Suitable alkylamine salts may be selected from the group consisting of choline, diethylamine, 2-diethylaminoethanol, N,N-dimethylethanolamine, tromethamine, ethanolamine, and diolamine.

Suitable heterocyclic amine salts may be selected from the group consisting of piperazine, pyrrolidine, morpholine, 1-(2-hydroxyethyl)-pyrrolidine, and 4-(2hydroxyethyl)-morpholine.

Quaternary ammonium salts can be formed, for example, by reaction with lower alkyl halides, such as methyl, ethyl, propyl, and butyl chlorides, bromides, and iodides, with dialkyl sulphates, with long chain halides, such as decyl, lauryl, myristyl, and stearyl chlorides, bromides, and iodides, and with aralkyl halides, such as benzyl and phenethyl bromides.

In an embodiment, the oral pharmaceutical composition comprises a salt of treprostinil selected from the group consisting of calcium, ethylenediamine, choline, TRIS, sodium, potassium, glucamine, benzathine, and procaine.

In yet another embodiment, suitable treprostinil salts including the potassium salt of treprostinil, the 1-arginine salt of treprostinil, the 1-lysine salt of treprostinil, the N-methylglucamine salt of treprostinil, the choline salt of treprostinil, the magnesium salt of treprostinil, the ammonium salt of treprostinil, the calcium salt of treprostinil and the tromethamine salt of treprostinil.

Representative pharmaceutically acceptable salts include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

In the manufacture of a composition according to the invention, hereinafter referred to as a “composition,” treprostinil, or its prodrug, or pharmaceutically acceptable salts thereof, may be admixed with, inter alia, a pharmaceutically acceptable carrier. The carrier must, of course, be acceptable in the sense of being compatible with all other ingredients in the formulation and must not be deleterious to the subject. The carrier may be a solid or a liquid, or either, and is preferably formulated with the treprostinil, a prodrug or a pharmaceutically acceptable salt as a unit-dose formulation, for example, a tablet, which may contain from 0.05% to 95% by weight of treprostinil.

Treprostinil, its prodrugs, or pharmaceutically acceptable salts thereof incorporated in the compositions of the invention may be prepared by any of the well-known methods in the art.

Process for preparation of treprostinil, treprostinil derivatives and intermediates useful in preparation of treprostinil are described in U.S. Pat. Nos. 4,306,075, 6,700,025, 6,809,223 and 6,765,117. U.S. Patent No. 4,306,075 (col. 40, I. 41-62) discloses a procedure for the preparation of pharmacologically acceptable salts of treprostinil, wherein preparation of an inorganic salt of treprostinil can be carried out by dissolution of treprostinil in water, followed by neutralization with appropriate amounts of a corresponding inorganic base.

In an embodiment, a process for preparing a treprostinil salt comprises the steps of: dissolving treprostinil in a water-miscible organic solvent to form a treprostinil solution, reacting the treprostinil solution with an aqueous basic solution containing an alkali metal cation to form a reaction mixture containing the treprostinil salt, allowing crystallization of the treprostinil salt, and collecting the treprostinil salt formed.

The oral compositions of the invention may be prepared by any suitable method of pharmacy which includes the step of bringing into association the active compound and a suitable pharmaceutically acceptable carrier (which may contain one or more accessory ingredients).

In general, the formulations of the invention are prepared by uniformly and intimately admixing treprostinil or its prodrug, or a pharmaceutically acceptable salt thereof with a liquid or finely divided solid carrier, or both, and then, if necessary, shaping the resulting mixture. For example, a tablet may be prepared by compressing or molding a powder or granules containing treprostinil or its prodrug, or a pharmaceutically acceptable salt thereof, optionally, with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made in a suitable machine by molding the powdered compound moistened with an inert liquid binder.

Compositions suitable for oral administration may be presented in unitary or discrete units, such as capsules, cachets, lozenges, or tablets, each containing a predetermined amount of treprostinil or its prodrug, or a pharmaceutically acceptable salt thereof; as a powder or granules; as a solution or a suspension in an aqueous or non-aqueous liquid; or as an oil-in-water or water-in-oil emulsion.

Preferably, the composition of the invention exhibit extended release of treprostinil, or a prodrug, or a pharmaceutically acceptable salt thereof.

The extended release composition can be prepared using rate controlling substances by various methods known in the art. In an embodiment, the composition comprises matrix of treprostinil or its prodrug, or a pharmaceutically acceptable salt thereof and rate controlling substances. In a further embodiment, the composition comprises a core comprising treprostinil or its prodrug, or a pharmaceutically acceptable salt thereof and a coating of rate controlling substances over said core. In a further embodiment, the composition may comprise a combination of a matrix and a coating type extended release system.

Various rate controlling substances suitable for use in the composition of the present invention may be selected from one or more of hydrophilic or hydrophobic polymers such as polyvinyl acetate, cellulose acetate, cellulose acetate butyrate, cellulose acetate propionate, ethyl cellulose, a fatty acid, a fatty acid ester, an alkyl alcohol, a wax, shellac, rosin, zein (prolamine from corn), povidone, kollidon SR, a poly(meth)acrylate, microcrystalline cellulose or poly(ethylene oxide), polyuronic acid salts, cellulose ethers, xanthan gum, tragacanth gum, gum karaya, guar gum, acacia, gellan gum, locust bean gum, alkali metal salts of alginic acid or pectic acid, sodium alginate, potassium alginate, ammonium alginate, hydroxypropyl cellulose, hydroxy ethyl cellulose, hydroxypropyl methyl cellulose, carboxyvinyl polymers, polymerized gelatin, shellac, methacrylic acid copolymer type C NF, cellulose butyrate phthalate, cellulose hydrogen phthalate, cellulose propionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethyl cellulose (CMEC), hydroxypropyl methylcellulose acetate succinate (HPMCAS), and acrylic acid polymers and copolymers like methyl acrylate, ethyl acrylate, methyl methacrylate and/or ethyl methacrylate with copolymers of acrylic and methacrylic acid esters (Eudragit NE, Eudragit RL, Eudragit RS), pH dependent polymers such as methacrylic acid copolymer type C (Eudragit L 100 55), methacrylic acid copolymer type C (Eudragit L 30D 55), methacrylic acid copolymer type A (Eudragit L 100), methacrylic acid copolymer type C (Eudragit S 100) and mixtures thereof.

In an embodiment, the oral pharmaceutical composition is in the form of an osmotic tablet.

In an embodiment, an osmotic delivery system or dosage form may comprise: (a) treprostinil, or a prodrug, or a pharmaceutically acceptable salt thereof excluding the diethanolamine salt; (b) at least one release enhancing agent; (c) at least one osmotic agent; (d) optionally, a binder to aid tableting; (e) optionally, at least one lubricant to aid the tableting process; (f) typically at least one coating polymer; and (g) optionally, at least one plasticizer.

Suitable release enhancing agents include wicking agents, such as high HLB surfactants (for example Tween 20, Tween 60 or Tween 80; ethylene oxide propylene oxide block copolymers (aka Pluronics®)), ionic surfactants such as sodium lauryl sulfate, sodium docusate, non-swelling hydrophilic polymers such as cellulose ethers, and polyethylene glycols (PEGs); complexing agents such as polyvinyl pyrrolidone, cyclodextrins and non-ionic surface active agents; and micelle forming agents, which may be surface active agents such as Tweens (Poly(ethylene oxide) modified sorbitan monoesters), Spans (fatty acid sorbitan esters), sodium lauryl sulfate, and sodium docusate.

The release enhancing agents are incorporated in the core tablet formula and constitute from 0.5% to 90% by weight of the formula, preferably from 1% to 50%. Most preferably, release-enhancing agents constitute from 1% to 20% by weight of the formulation. They can be incorporated during granulation or post-granulation. The release enhancing agent(s) can be added in the form of solid powder or can be dissolved in the granulating liquid and sprayed during granulation. In order to achieve intimate mixing with the drug(s) the release enhancing agent can be mixed with the drug alone before the other excipients are incorporated. Alternatively, the release enhancing agent(s) and the drug can be dissolved in the granulating liquid and sprayed during granulation. In yet another way, the release enhancing agent(s) and the drug can be dissolved in a solvent, and when the solution is dried, the solid mass thus obtained can be milled and then mixed with the rest of the excipients for further processing.

Osmotic agents are well known to those skilled in the art. Osmotic agents suitable for the present invention are simple sugars such as sucrose, xylitol, glucose, lactose; salts such as sodium chloride, potassium chloride; low molecular weight hydrophilic polymers such as cellulose ethers, maltodextrins, and cyclodextrins. Osmotic agents can be incorporated in the composition of this invention in the amount of from 1% by weight to 90% by weight, preferably from 5% to 80% and most preferably from 10% to 80% by weight. Osmotic agents are typically incorporated in the formulation during granulation.

The granules can also be blended with other excipients as needed to aid the manufacturing of the desired dosage form: tablets, capsules or pellets. Tablets are compressed on a standard rotary tablet press.

The core tablet is typically coated with a semi-permeable membrane containing at least one plasticizer. The coating polymer is dissolved with at least one plasticizer in an appropriate solvent or a mixture of solvents and sprayed on the tablets for coating. The coating polymers include, but are not limited, to cellulose acetate. The coating membrane preferably also contains at least one plasticizer to improve flexibility and durability of the coat. Such plasticizers include, but are not limited to, triethyl citrate (TEC), propylene glycol(PG), or mixtures thereof in ratios of TEC to PG ranging from 25:75 to 75:25; Tween 80, polyethylene glycols (PEGs); other polyoxyethylene sorbitan esters, triacetin, diethyl phthalate, mineral oil, tributyl sebacate, and glycerol. The coating level can vary from 1% to 25%, preferably from 2% to 20%, and most preferably from 3% to 10% by weight.

The semi-permeable wall also includes at least one opening to provide for the osmotic delivery of the drug(s). In general, the at least one opening has a diameter of from 50μm to 1000μm, preferably from 100μm to 800μm. The opening is formed by drilling using a laser or any other appropriate hole drilling system. The opening can be of any shape. The various shapes contemplated include, but are not limited to round, cross-shaped, rectangular, diamond, star, and square shapes. As the dosage unit (osmotic tablet) imbibes water, the release-enhancing agents go into solution providing an environment for the drug to dissolve. The osmotic agent(s) in the core tablet draws water into the core tablet creating an osmotic gradient across the semi-permeable membrane. The osmotic gradient pushes the drug in the solution out through the laser-drilled hole.

In an embodiment, the composition containing the appropriate release-enhancing agents can be prepared, e.g., in the form of capsules or pellets. Once the capsule is filled with the appropriate combination of drugs, release-enhancing agents and excipients, it is coated with a semi-permeable membrane. At least one opening is drilled through the semi-permeable membrane to allow drug release.

Formulations suitable for buccal (sub-lingual) administration include lozenges comprising treprostinil or its prodrug, or a pharmaceutically acceptable salt thereof, in a flavoured base, usually sucrose and acacia or tragacanth; and pastilles comprising the compound in an inert base such as gelatin and glycerine or sucrose and acacia.

Pharmaceutical compositions described herein may be used alone or in combination with other compounds. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the composition of the invention and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present invention.

This invention further provides use of the oral pharmaceutical composition of the invention for promoting vasodilation, inhibiting platelet aggregation and thrombus formation, stimulating thrombolysis, inhibiting cell proliferation (including vascular remodeling), providing cytoprotection, preventing atherogenesis and inducing angiogenesis. The compositions of the present invention may be used in the treatment of/for one or more of the following: pulmonary hypertension, ischemic diseases (e.g., peripheral vascular disease, Raynaud's phenomenon, Scleroderma, myocardial ischemia, ischemic stroke, renal insufficiency), heart failure (including congestive heart failure), conditions requiring anticoagulation (e.g., post MI, post cardiac surgery), thrombotic microangiopathy, extracorporeal circulation, central retinal vein occlusion, atherosclerosis, inflammatory diseases (e.g., COPD, psoriasis), hypertension (e.g., preeclampsia), reproduction and parturition, cancer or other conditions of unregulated cell growth, cell/tissue preservation and other emerging therapeutic areas where prostacyclin treatment appears to have a beneficial role. When the composition includes other cardiovascular agents (e.g., calcium channel blockers, phosphodiesterase inhibitors, endothelial antagonists, antiplatelet agents) the composition may demonstrate additive or synergistic benefits.

In an embodiment, a method of reducing the side effects or toxicity associated with oral administration of a composition of treprostinil or a salt thereof, the method comprising orally administering the pharmaceutical composition of treprostinil, prodrug, or pharmaceutically acceptable salt thereof, excluding the diethanolamine salt.

In a further embodiment, the invention relates to a method of providing treatment for PAH that is comparable to a method of treating PAH that consists essentially of intravenous or inhalation administration of treprostinil or a pharmaceutically acceptable salt thereof. The comparable method consists essentially of orally administering the pharmaceutical composition comprising treprostinil, a prodrug, or a pharmaceutically acceptable salt thereof, excluding the diethanolamine salt.

What is claimed is:

1. An oral pharmaceutical composition comprising treprostinil free acid, a prodrug or a pharmaceutically acceptable salt thereof excluding the diethanolamine salt, and one or more pharmaceutically acceptable carriers.

2. The composition of claim 1, wherein an oral bioavailability of treprostinil, a prodrug or a pharmaceutically acceptable salt thereof in said composition is greater than 10%.

3. The composition of claim 1, wherein an oral bioavailability of treprostinil, a prodrug or a pharmaceutically acceptable salt thereof in said composition is at least or greater than 17%.

4. The composition of claim 1, wherein said pharmaceutically acceptable salt is selected from the group consisting of calcium, ethylenediamine, choline, hydroxymethyl aminomethane (TRIS), sodium, potassium, glucamine, benzathine, and procaine.

5. The composition of claim 4, wherein said pharmaceutically acceptable salt is sodium.

6. The composition of claim 1, wherein an oral bioavailability of said composition is equal to or greater than the oral bioavailability of an treprostinil diethanolamine composition having the same amount of treprostinil.

7. The composition of claim 1, wherein said composition is in the form of a solid unit dosage form.

8. The composition of claim 1, wherein said composition is in the form of an extended release solid unit dosage form.

9. The composition of claim 1, wherein said composition is in the form of an osmotic tablet.

10. The osmotic tablet of claim 9, wherein an oral bioavailability of treprostinil, a prodrug or a pharmaceutically acceptable salt thereof in said tablet is greater than 10%.

11. A method of treating pulmonary hypertension, ischemic diseases, heart failure, conditions requiring anticoagulation, thrombotic microangiopathy, extracorporeal circulation, central retinal vein occlusion, atherosclerosis, inflammatory diseases, hypertension, reproduction and parturition, cancer or other conditions of unregulated cell growth, cell/tissue preservation in a patient comprising orally administering the composition of claim 1 to said patient.

12. A method of reducing the side effects or toxicity associated with oral administration of the composition of treprostinil or a salt thereof in patient comprising orally administering the composition of claim 1 to said patient.

13. A method of converting a treatment for pulmonary arterial hypertension in a patient, the method consisting essentially of intravenous administration of the composition of claim 1 to said patient.

14. A method of converting a treatment for pulmonary arterial hypertension in a patient, the method consisting essentially of orally administering the composition of claim 1 to said patient.
